(19) United States
(12) Patent Application Publication
(10) Pub. No.: US 2015/0013609 A1
(43) Pub. Date: Jan. 15, 2015

(54) COMPOSITIONS AND METHODS FOR CAUSING, ENHANCING, AND/OR EXPEDITING BIODEGRADATION OF ARTICLES

(71) Applicant: Wanda M. Weder and William F. Straeter, not individually but solely as Trustees of The Family, Highland, IL (US)

(72) Inventor: Donald E. Weder, Highland, IL (US)

(21) Appl. No.: 13/940,993

(22) Filed: Jul. 12, 2013

(51) Int. Cl. A01K 67/033

(52) U.S. Cl. CPC A01K 67/033
USPC 119/6.5; 119/6.7

(57) ABSTRACT

Kits and assemblies for causing, enhancing, and/or expediting consumption of an article by at least one living organism are disclosed. Methods of producing and using same are also provided.

### CROSS REFERENCE TO RELATED APPLICATIONS/INCORPORATION BY REFERENCE STATEMENT

Not Applicable.

### STATEMENT REGARDING FEDERALLY SPONSORED RESEARCH AND DEVELOPMENT

Not Applicable.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is a perspective view of one embodiment of a kit constructed in accordance with the presently disclosed and claimed inventive concept(s).

FIG. 2 is a perspective view of a second embodiment of a kit constructed in accordance with the presently disclosed and claimed inventive concept(s).

FIG. 3A is a perspective view of another embodiment of a kit constructed in accordance with the presently disclosed and claimed inventive concept(s).

FIG. 3B is a cross-sectional view of the kit of FIG. 3A taken along lines 3B-3B.

FIG. 4 is a perspective view of another embodiment of a kit constructed in accordance with the presently disclosed and claimed inventive concept(s).

FIG. 5 is a perspective view of yet another embodiment of a kit constructed in accordance with the presently disclosed and claimed inventive concept(s).

FIG. 6 is a bottom plan view of a further embodiment of a kit constructed in accordance with the presently disclosed and claimed inventive concept(s).

FIG. 7 is a perspective view of yet another embodiment of a kit constructed in accordance with the presently disclosed and claimed inventive concept(s).

FIG. 8 is a perspective view of an embodiment of an assembly constructed in accordance with the presently disclosed and claimed inventive concept(s), wherein the assembly may be utilized alone or in combination with any of the embodiments of kits described herein.

FIG. 9 contains a photograph of one embodiment of an article (i.e., a flower pot cover) of a kit constructed in accordance with the presently disclosed and claimed inventive concept(s).

FIGS. 10-12 contain photographs of the flower pot cover of FIG. 9 having soil and earthworms disposed therein, wherein the earthworms were either contained in the kit, or the kit contained instructions for obtaining the earthworms.

FIGS. 13-21 contain photographs illustrating various stages of consumption of the flower pot cover of FIG. 9 by the earthworms disposed therein, as shown in FIGS. 10-12.

FIG. 22 contains a photograph of another embodiment of flower pot covers, the flower pot covers being contained in at least one kit constructed in accordance with the presently disclosed and claimed inventive concept(s).

FIG. 23 contains a photograph of four flower pot covers of FIG. 22 divided into smaller pieces, moistened, and placed in a container with earthworms, wherein the earthworms were either contained in the kit, or the kit contained instructions for obtaining the earthworms.

FIGS. 24-25 contain photographs illustrating various stages of consumption of the flower pot covers of FIGS. 22-23 by the earthworms.

FIG. 26 contains a photograph of yet another embodiment of an article (i.e., a decorative grass/packing material) of a kit constructed in accordance with the presently disclosed and claimed inventive concept(s).

FIG. 27 contains a photograph illustrating the results of consumption of a decorative grass/packing material like the decorative grass/packing material of FIG. 26 by earthworms, wherein the earthworms were either contained in the kit, or the kit contained instructions for obtaining the earthworms.

### DETAILED DESCRIPTION

Before explaining at least one embodiment of the presently disclosed and claimed inventive concept(s) in detail, it is to be understood that the presently disclosed and claimed inventive concept(s) is not limited in its application to the details of construction and the arrangement of the components or steps or methodologies set forth in the following description or illustrated in the drawings. The presently disclosed and claimed inventive concept(s) is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles and/or methods disclosed and claimed herein can be made and executed without undue experimentation in light of the present disclosure. While the articles and methods of the presently disclosed and claimed inventive concept(s) have been described in terms of preferred embodiments, it will be apparent to those of skill in the art that variations may be applied to the articles and/or methods and in the steps or in the sequence of steps of the method described herein without departing from the concept, spirit, and scope of the presently disclosed and claimed inventive concept(s). All such similar substitutes and modifications apparent to those skilled in the art are deemed to be within the spirit, scope, and concept of the presently disclosed and claimed inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

The term “living organism” as used herein will be understood to include any organism capable of consuming the articles disclosed herein by biodegradative means. Non-limiting examples of living organisms contemplated for use in accordance with the presently disclosed and claimed inventive concept(s) include worms (such as but not limited to, earthworms), termites, ants, microbes (including but not limited to, bacteria, viruses, protozoa, algae, fungi, and the like).

The terms “microbe” and “microorganism” may be utilized interchangeably herein.

The combination of terms “nutrients,” “attractants,” “stimulants,” and “hormones” encompass any substance for which a living organism utilized herein has an affinity. For example but not by way of limitation, these substances may provide nutrition to the organism, attract the organism, and/or stimulate the organisms' metabolism and/or consumption rate, and/or function in any other manner that would be conducive to the consumption of an article by the living organism.

The term “associate” as used herein will be understood to refer to the direct or indirect connection of two or more items.

Turning now to the presently disclosed and claimed inventive concept(s), one embodiment thereof is directed to a kit that includes an article formed of a material, at least a portion of which is consumable by at least one living organism. The kit may further include at least one living organism capable of consuming the consumable portion of the material, or instructions for obtaining at least one living organism capable of consuming the consumable portion of the material from which the article is produced. In addition, the kit may further include instructions for causing the at least one living organism to begin consumption of the consumable portion of the article.

Any article for which consumption of at least a portion thereof is desired may be utilized in accordance with the presently disclosed and claimed inventive concept(s). In particular, the degradation/consumption of disposable articles, and in particular, disposable paper articles, is typically desired and thus encompassed within the scope of the presently disclosed and claimed inventive concept(s). Non-limiting examples of articles include various types of floral packaging materials (including but not limited to, a sheet of wrapping material, a roll of wrapping material, a bag, a sleeve, combinations thereof, and the like); decorative grasses and other types of packaging materials; cups, plates, bowls, and other types of eating/drinking supplies; food wrappers and food containers; bags; as well as various paper products like toilet paper and paper towels; as well as any combination thereof.

The consumable portion of the article may be formed of any material capable of consumption by at least one living organism. Non-limiting examples of consumable materials that may be utilized herein include any of the renewable and/or biodegradable materials described or otherwise contemplated herein. Particular non-limiting examples of consumable materials that may be utilized in accordance with the presently disclosed and claimed inventive concept(s) include paper, cardboard, similar-type consumable materials, as well as any combination thereof.

Any living organism capable of consuming material as described or otherwise contemplated herein for the purpose of biodegradation of the material falls within the scope of the presently disclosed and claimed inventive concept(s). Non-limiting examples of organisms capable of functioning in accordance with the presently disclosed and claimed inventive concept(s) include worms, termites, ants, microorganisms, and any combination thereof.

The living organisms utilized in accordance with the presently disclosed and claimed inventive concept(s) may be utilized at any point during their life cycle. For example but not by way of limitation, eggs, embryos, youth, and/or adult organisms may be utilized. In addition, the living organisms (regardless of life stage) may be disposed in a package, covering, or the like. The package/covering may be naturally-occurring, such as but not limited to, a cocoon or spore (a non-limiting example of which is an earthworm cocoon). Optionally, a package/covering may be added to the living organism; for example but not by way of limitation, a living organism may be encapsulated. In addition, it may be desirable to encapsulate a living organism that is already disposed within a naturally-occurring package/covering.

A non-limiting example of living organisms contemplated for use herein are earthworms, such as those typically present in gardens and flower beds, including but not limited to the species Lumbricus terrestris, Eisenia fetida, and/or Eisenia hortensis. Another non-limiting example of earthworms that may be utilized in accordance with the presently disclosed and claimed inventive concept(s) include the VERMIPOD™ encapsulated earthworm cocoons (VermiPod, LLC., Highland, Ill.). Production of encapsulated earthworm cocoons is described in detail in U.S. Pat. No. 6,834,614, issued to Kreitzer on Dec. 28, 2004, the entire contents of which are expressly incorporated herein by reference.

Other examples of living organisms possessing biodegradative abilities are well known in the art. Based on the disclosure provided herein, it is well within the skill of a person of ordinary skill in the art to select a particular type of living organism for use in the kits and methods described herein, and thus no further description thereof is deemed necessary.

The use of combinations of living organisms also falls within the scope of the presently disclosed and claimed inventive concept(s). One such combination includes multiple species of living organisms. A non-limiting example of such a combination includes earthworms with at least one microbe (such as bacteria); however, any combination of species of living organisms described or otherwise contemplated herein may be utilized (including two or more species, three or more species, four or more species, five or more species, as well as higher combinations of species). In addition, combinations of living organisms at different stages in their life cycles may be utilized. Also, combinations of living organisms in which only a portion thereof contain a naturally-occurring packaging/covering may be utilized herein, as well as combinations of encapsulated and non-encapsulated living organisms. Further, any combination of the above combinations (such as but not limited to, multiple species of living organisms, each present at more than one stage in their life cycle and in both encapsulated and non-encapsulated forms) is encompassed by the scope of the presently disclosed and claimed inventive concept(s).

It may be desirable to provide the living organism(s) in a “biodegradably inactive form”, so that consumption of the article does not begin immediately upon contact with the living organism(s). Rather, it may be desired that the living organism(s) remain in a somewhat dormant state until “activated” by contact with a particular agent (such as but not limited to, water and/or soil). Once the living organism(s) is brought into contact with the particular agent, the living organism(s) become biodegradably active and begin consumption of the article. In a non-limiting embodiment, the living organism(s) may be rendered biodegradably inactive by the package/covering disposed thereabout (such as but not limited to, a capsule or cocoon).

In certain embodiments, the living organism(s) may be separate from and unattached to the article. Alternatively, the living organism(s) may be attached to and/or contained within the article. The living organism(s) may be directly and/or indirectly attached to the article by any method known in the art or otherwise contemplated herein. For example but not by way of limitation, a bonding material may be utilized to directly attach the living organism(s) to the article. Alternatively, a coating containing the living organism(s) may be disposed on a surface of the article. A non-limiting example of a type of coating that may be utilized in accordance with the presently disclosed and claimed inventive concept(s) is a lacquer.

The article may be formed of a material, or have a composition disposed thereon and/or therein, that functions to attract and/or nourish the living organism(s) and/or enhance and/or expedite the rate of consumption of the material by the living organism(s). Non-limiting examples of compositions that may be utilized in this manner include nutrients, stimulants, attractants, hormones, like substances, and any combination thereof. Particular non-limiting examples of substances that may be present in the composition include a metal, a metal salt, a metal stearate, theobromine, theophylline, mildronate, 3-(2,2,2-trimethylhydraziniumyl)propionate, caffeine, starch, sugar, tea, tea leaves, chocolate, cocoa, coffee, coffee grounds (i.e., coffee grounds used to make a coffee beverage), ground coffee beans, a short chain saturated fat, a grain, a thyroid hormone, a pheromone, dichloroacetic acid (DCA), sodium dichloroacetate, an enzyme, and combinations thereof.

In a particular embodiment of the presently disclosed and claimed inventive concept(s), the kit comprises a flower pot cover having a consumable portion that is formed of paper, and at least one worm.

In certain embodiments, separate and/or unattached elements of the kit may be associated with one another via a packaging. The term “packaging” as used herein will be understood to include any element(s) that associate the content(s) of the kits contemplated herein with one another. Non-limiting examples of packaging that may be utilized in accordance with the presently disclosed and claimed inventive concept(s) include a bag, a wrapper, adhesive, cohesive, tape, a flexible container, a rigid container, stretch wrap, shrink wrap, and combinations thereof.

Any of the kits described or otherwise contemplated herein may contain further separate components for use in the methods described herein below. For example but not by way of limitation, the kit may include one or more materials in which the article may be disposed to provide an environment conducive to the article's degradation by the living organism(s). Alternatively, the kit may include one or more compositions as described herein above that function to attract and/or nourish the living organism(s) and/or enhance and/or expedite the rate of consumption of the article by the living organism(s). In this manner, the composition may be applied to the article when at a time when consumption thereof is desired.

Other embodiments of the presently disclosed and claimed inventive concept(s) include methods of degrading an article using any of the kits described herein above. In the method, the kit is opened, and if the living organism(s) is not contained within the kit, the living organism(s) is obtained as described in the provided instructions. The article is then exposed to the living organism(s), and the living organism(s) consumes the consumable portion of the article. As the living organism(s) consumes the article, the living organism(s) may convert the article to soil. Alternatively or in addition, the living organism(s) may produce excretions (such as but not limited to, earthworm castings) that add various desirable features to soil that is added to the method and/or produced by the living organism(s). These features include, but are not limited to, humus, nitrogen, phosphates, potassium, plant growth hormones, and factors enhancing soil water retention properties.

The article may be directly subjected to consumption in its originally provided form, or the article may be divided into smaller pieces/segments (such as but not limited to, by cutting or tearing) for ease and/or expedition of consumption thereof. In addition, the method may further include placing the article in an environment conducive to biodegradation thereof. For example but not by way of limitation, worms typically require moisture for consumption of an article, so the article may be moistened to provide an environment conducive to its degradation. Optionally, the article may be placed in contact with soil and/or a composter in order to provide a desirable environment for degradation.

Also, the method may further include the step of activating an organism to begin consumption of the article. The activation step may include removal of a package/covering from an organism to begin consumption of the article. Alternatively, activation of the organism may require disposal in the environment conducive to biodegradation as described above.

Further, the method may further include the step of disposing on the article (or otherwise placing the article in contact with) one or more compositions as described herein above that function to attract and/or nourish the living organism(s) and/or enhance and/or expedite the rate of consumption of the article by the living organism(s). In this manner, placing the article in contact with the composition may result in (1) activation of the living organism(s) to begin consumption of the article, (2) attraction and/or nourishment of the living organism(s) that are consuming the article, and/or (3) enhancement and/or expedition of the current rate of consumption of the article by the living organisms(s).

Instructions for determining the ideal consumption/biodegradation conditions to utilize with the VERMIPOD™ encapsulated earthworm cocoons (VermiPod, LLC., Highland, Ill.) are described in detail in U.S. Pat. No. 7,478,604, issued to Kreitzer on Jan. 20, 2009, the entire contents of which are expressly incorporated herein by reference. The ideal consumption/biodegradation conditions for other biodegradative organisms that can be utilized in accordance with the presently disclosed and claimed inventive concept(s) are well known in the art and/or can easily be determined by a person having ordinary skill in the art, given the description provided herein. Therefore, no further description of determining these conditions is deemed necessary.

Another embodiment of the presently disclosed and claimed inventive concept(s) includes an assembly. The assembly includes an article having at least a portion thereof formed of a material consumable by at least one living organism; non-limiting examples of articles and materials have been described in detail herein above. The assembly also includes a composition disposed on and/or in at least a portion of the material that functions to attract and/or nourish the living organism(s) and/or enhance and/or expedite the rate of consumption of the treated material by the living organism(s). The composition may be a nutrient, a stimulant, an attractant, a hormone, and/or a combination thereof, as described in detail herein above. The composition may enhance and/or expedite consumption of the treated material by the living organism(s) by any method known in the art or otherwise contemplated herein. Non-limiting examples of enhancing/expediting methods include: (a) attracting the at least one living organism to the article; (b) increasing the speed of consumption of the article by the at least one living organism; (c) increasing activity of the at least one living organism; and (d) enhancing the health of the at least one living organism. The composition may be disposed on a surface of the material and/or impregnated within the material.

In another embodiment of an assembly constructed in accordance with the presently disclosed and claimed inventive concept(s), an assembly similar to the assembly described herein above is provided, except that the article is formed of a substantially non-biodegradable material, and the composition disposed on and/or in at least a portion of the material functions to cause its consumption by the at least one living organism. In this embodiment, the composition may only be disposed on a portion of the material, and the remaining portion of the material is not substantially consumed by the at least one living organism.

In yet another embodiment of the presently disclosed and claimed inventive concept(s), a method of enhancing and/or expediting biodegradation of an article is provided. In the method, a composition is disposed on and/or in at least a portion of a material from which an article is formed (the article, material, and composition being described in detail herein above), wherein at least a portion of the material is consumable by at least one living organism, and wherein the composition enhances and/or expedites consumption of the treated material by the at least one living organism.

In yet a further embodiment, a method of producing a biodegradable article formed from a substantially non-biodegradable material is provided, in which a composition is disposed on and/or in at least a portion of a substantially non-biodegradable material from which an article is formed (the article, material, and composition being described in detail herein above), wherein the composition enables consumption of the treated portion of the material by at least one living organism.

The above-described methods may further include the step of contacting the article (containing the composition that causes, expedites, and/or enhances biodegradation thereof) with at least one living organism to begin consumption thereof. The methods may further include any of the other steps described in detail herein above with reference to the methods of using the kits constructed in accordance with the presently disclosed and claimed inventive concept(s) (i.e., placing the article and organism(s) in an environment conducive to consumption of the article, dividing the article into smaller pieces/segments, moistening the article, activating the organism(s), etc.).

While examples of sequences of method steps are provided herein, it is to be understood that the method steps, as well as the order of the steps, may vary. Any combination of method steps and sequences thereof may be utilized so long as the kits and/or assemblies described herein are capable of functioning in accordance with the presently disclosed and claimed inventive concept(s).

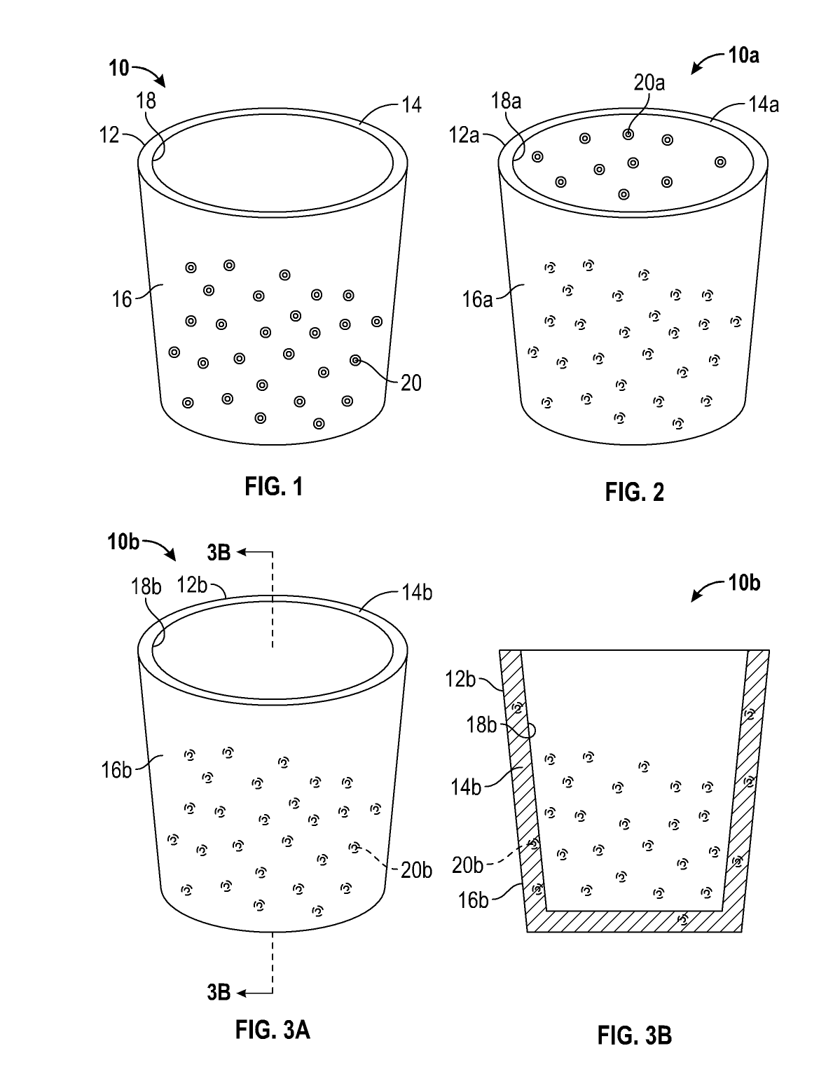

Turning now to the Drawings, shown in FIG. 1 is an embodiment of a kit 10 constructed in accordance with the presently disclosed and claimed inventive concept(s). The kit 10 includes an article 12 that has a sidewall 14 with an outer peripheral surface 16 and an inner peripheral surface 18. At least a portion of the article 12 is formed of a material that is consumable by at least one living organism. The kit 10 further includes a plurality of living organisms 20 disposed on the outer peripheral surface 16 of the article 12. The living organisms 20 may be directly attached to the outer peripheral surface 16 of the article 12; for example but by way of limitation, a bonding material may be used to attach the living organisms 20 to the outer peripheral surface 16 of the article 12, or the living organisms 20 may possess an adhesive characteristic (such as an inherent property thereof or a property provided by an encapsulation material) that attaches the living organism 20 to the outer peripheral surface 16 of the article 12. Optionally, a coating (such as, but not limited to, a lacquer) containing the living organisms 20 may be disposed on the outer peripheral surface 16 of the article 12 to attach the living organisms 20 to the outer peripheral surface 16 of the article 12.

While a plurality of living organisms 20 is depicted in FIG. 1, it should be understood that the disposal of a single living organism 20 on the outer peripheral surface 16 of the article 12 is also encompassed by the presently disclosed and claimed inventive concept(s). In addition, when a plurality of living organisms 20 is disposed on the outer peripheral surface 16 of the article 12, the plurality of living organisms 20 may include a single type/species of organism or any combination of species and/or types of organisms, as described in greater detail herein. When a plurality of living organisms 20 is present (whether a single type/species or various combinations of species and/or types of organisms), the living organisms 20 may be disposed on the outer peripheral surface 16 of the article 12 in any amount or density. The living organisms 20 may be disposed on the outer peripheral surface 16 of the article 12 in a seemingly random arrangement, or the living organisms 20 may be disposed on the outer peripheral surface 16 of the article 12 in a controlled manner that provides a desired density of living organisms 20 present on the surface area of the outer peripheral surface 16 of the article 12. Methods of determining appropriate surface area densities for the living organisms are within the skill of person of ordinary skill in the art, and therefore no further discussion is considered necessary. The only requirement is that the living organism(s) 20 be disposed at a surface area density sufficient to allow consumption of the consumable portion of the article 12 by the living organism(s) 20.

The visibility of the organisms 20 on the outer peripheral surface 16 of the article 12 in FIG. 1 is only for the purpose of illustration of the contents of the kits contemplated herein. It is to be understood that the organisms 20 may be of a size that is visible on a surface, or the organisms 20 may be small enough that they are not substantially visible to the naked eye. The organisms 20 may also be provided with a covering/packaging disposed thereon of the same color as the outer peripheral surface 16 of the article 12, so that their presence on the outer peripheral surface 16 of the article is minimized or substantially eliminated.

FIG. 2 depicts a kit 10a that is similar to the kit 10 of FIG. 1 as described herein above. The kit 10a includes an article 12a having a sidewall 14a with an outer peripheral surface 16a and an inner peripheral surface 18a. The kit 10a further includes a plurality of living organisms 20a. The kit 10a differs from kit 10 in that the living organisms 20a are disposed on the inner peripheral surface 18a of the article 12a. Otherwise, the kit 10a is identical in construction and functions in the same manner as described herein above with reference to kit 10.

FIGS. 3A and 3B depict a kit 10b that is similar to the kits 10 and 10a of FIGS. 1 and 2, respectively. The kit 10b includes an article 12b having a sidewall 14b with an outer peripheral surface 16b and an inner peripheral surface 18b. The kit 10b further includes a plurality of living organisms 20b. The kit 10b differs from kits 10 and 10a in that the living organisms 20b are disposed within the sidewall 14b of the article 14b. Otherwise, the kit 10b is identical in construction and functions in the same manner as described herein above with reference to kits 10 and 10a.

While FIGS. 1, 2, 3A, and 3B depict three different ways in which living organisms may be disposed on or in an article, it is to be understood that combinations of any of the manners of disposal depicted in these Figures are also contemplated herein and thus fall within the scope of the presently disclosed and claimed inventive concept(s). That is, living organism(s) may be disposed on and/or in an article by any combination of two of (a) disposed on an outer peripheral surface of an article, (b) disposed on an inner peripheral surface of an article, and (c) disposed within a sidewall of an article. Alternatively, living organism(s) may be disposed on both the inner and outer peripheral surfaces of an article as well as within a sidewall of an article.

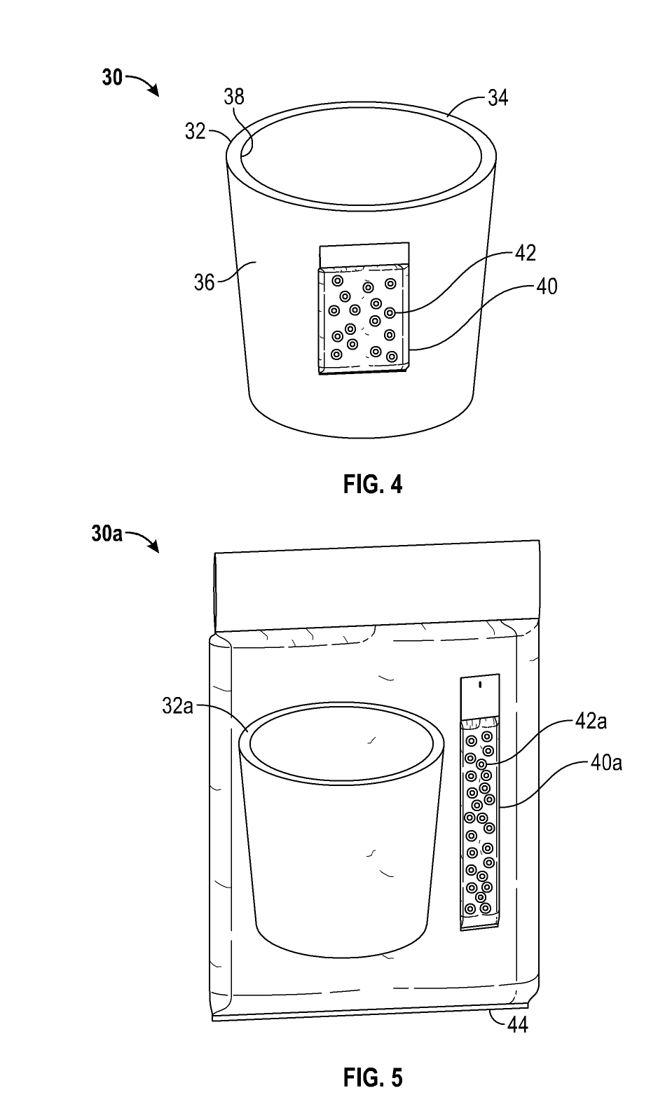

FIG. 4 depicts another embodiment of the kits of the presently disclosed and claimed inventive concept(s). A kit 30 includes an article 32 having a sidewall 34 with an outer peripheral surface 36 and an inner peripheral surface 38. The kit 30 further includes a package 40 containing a plurality of living organisms 42. While the kits 10, 10a, and 10b have living organisms 20, 20a, and 20b disposed thereon or therein, the kit 30 provides the living organisms 40 separate from the article 32 and within a package 40 that may be stored until degradation of the article 32 is desired. In this manner, the article 32 may be utilized in their normal manner without having the living organisms 40 disposed thereon and/or therein. After the article 32 has been utilized for its intended purpose, and biodegradation of the article 32 is desired, the package 42 may be opened, and the living organisms 40 placed in contact with the article 32 to begin consumption of the consumable portion of the article 32.

The package 40 is depicted as being detachably associated with the article 32 so that no further packaging is required. FIG. 5 depicts a kit 30a that is similar to the kit 30 of FIG. 4, except that an article 32a and a package 40a containing a plurality of living organisms 42a are disposed in packaging 44 that maintains the article 32a and the package 40a in association with one another.

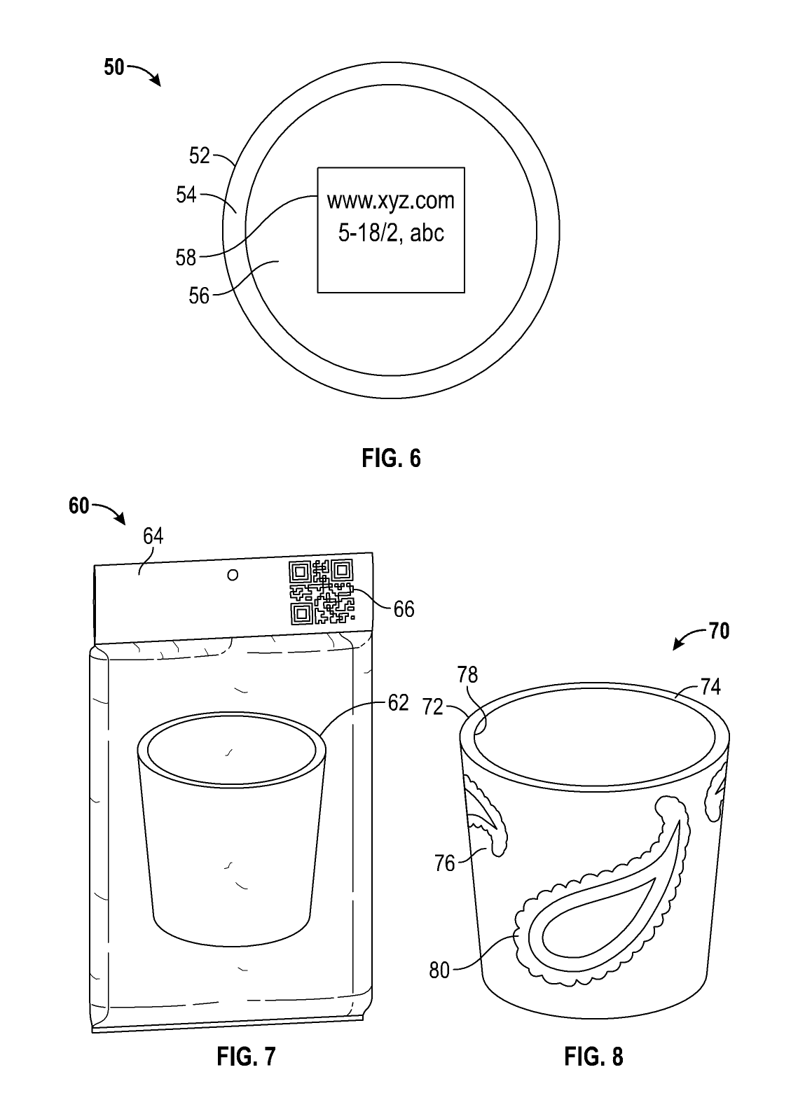

FIG. 6 depicts another embodiment of the kits of the presently disclosed and claimed inventive concept(s). A kit 50 includes an article 52 having a sidewall 54 and a bottom 56. While the previously described kits contained living organisms, the kit 50 contains instructions 58 for obtaining at least one living organism that is capable of consuming a consumable portion of the article 52. The instructions 58 may be disposed on any portion of the article 52, and disposal of the instructions 58 on the bottom 56 of the article 52 is for illustrative purposes only. The instructions 58 may be disposed on the article 52 by any manner known in the art, including but not limited to, printing and/or embossing a portion of the article 52, or by attachment of a label containing the instructions 58.

Optionally, the instructions for obtaining at least one living organism may not be attached directly to the article. FIG. 7 depicts a kit 60 that includes an article 62 contained within packaging 64 that has instructions 66 for obtaining at least one living organism disposed thereon. However, the disposal of the instructions 66 on the packaging 64 is for purposes of example only; it is to be understood that instructions for obtaining a living organism(s) may be associated with an article by any method known in the art, and thus any type of association of the article and instructions is encompassed within the scope of the presently disclosed and claimed inventive concept(s). For example but not by way of limitation, when the article is a consumable flower pot cover disposed about a flower pot containing a potted plant, the instructions may be placed upon a standard pick that is inserted into the soil of the potted plant. In a similar manner, a consumable floral wrapping having a floral grouping disposed therein may have a similar pick inserted into the floral grouping or a label attached thereto.

Any type of written wording and/or pictorial drawings capable of conveying a method of obtaining the living organism(s) may be utilized as the instructions described and claimed herein. Non-limiting examples of instructions for obtaining the at least one living organism that may be utilized as the instructions 58 and 66 include a website and/or e-mail address for placing an online order, a post card for requesting the organism through the mail, a bar code (such as but not limited to, a QR code) that is readable by an imaging device/code reader, instructions for gathering naturally-occurring organisms from their natural habitat, and any combination thereof.

FIG. 8 illustrates yet another embodiment of the presently disclosed and claimed inventive concept(s). FIG. 8 depicts an assembly 70 that includes an article 72 having a sidewall 74 with an outer peripheral surface 76 and an inner peripheral surface 78. The assembly 70 further comprises a composition 80 disposed on and/or in at least a portion of the article 72. The disposal of the composition 80 on the outer peripheral surface 76 of the article 72 is for purposes of illustration only, and it is to be understood that the composition may be disposed on the inner peripheral surface 78, or within the sidewall 74, or any combination of the inner and outer peripheral surfaces 76 and 78 and within the sidewall 74. The composition 80 causes, expedites, and/or enhances consumption of the treated portion of the article 72 by a living organism.

The composition 80 may be disposed on and/or in a portion of the article 72 or on and/or in substantially all of the article 72. The composition 80 may be disposed in any desired coating, shape, and/or pattern, and the fanciful pattern in which the composition 80 is illustrated as being disposed in FIG. 8 is for purposes of illustration only.

The assembly 70 of FIG. 8 may be utilized as any of the articles 12, 12a, 12b, 32, 32a, 52, and/or 62 of FIGS. 1-7. That is, the articles 12, 12a, 12b, 32 and/or 32a may be provided with both the living organism(s) 20, 20a, 20b, 42, and/or 42a disposed thereon or in combination therewith as well as a composition (like composition 80 of FIG. 8) disposed thereon; in a similar fashion, the articles 52 and/or 62 may contain instructions 58 and/or 66 as well as a composition similar to composition 80 disposed thereon.

### EXAMPLES

Examples are provided hereinbelow. However, the presently disclosed and claimed inventive concept(s) is to be understood to not be limited in its application to the specific experimentation, results and laboratory procedures. Rather, the Examples are simply provided as one of various embodiments and are meant to be exemplary, not exhaustive.

### Example 1

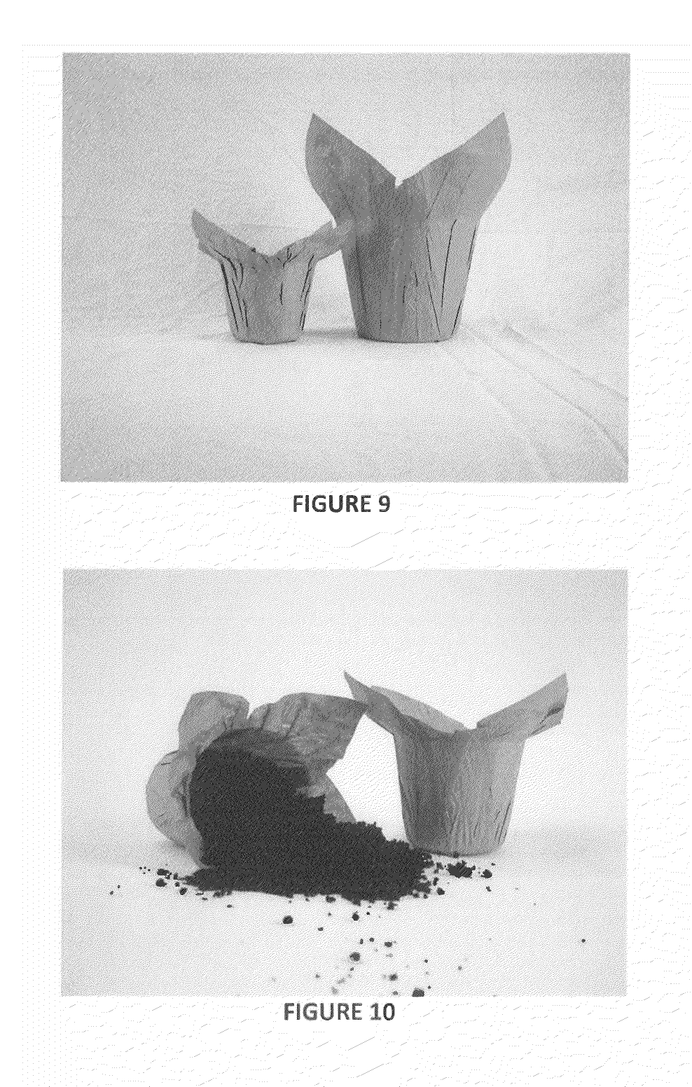

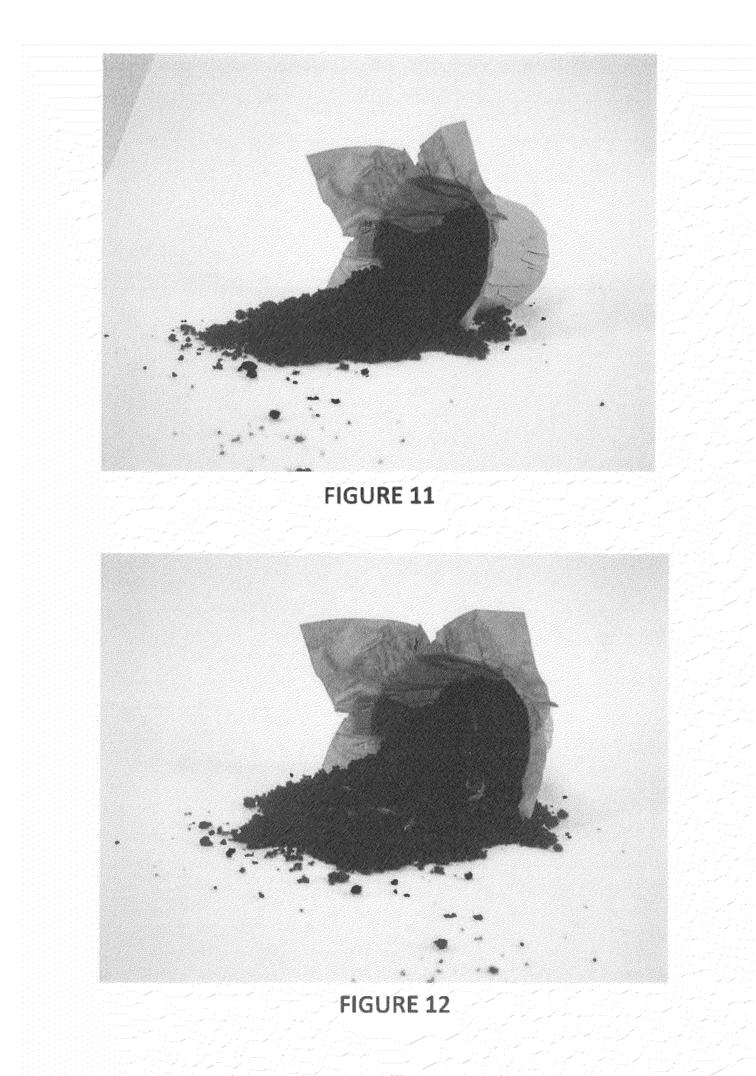

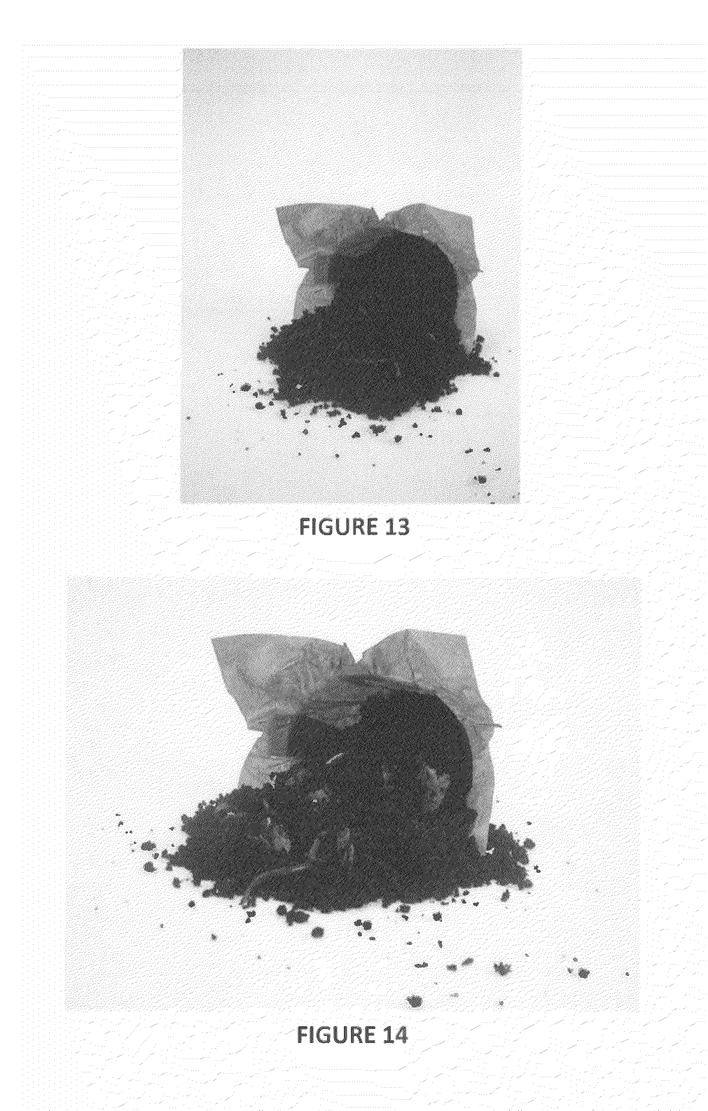

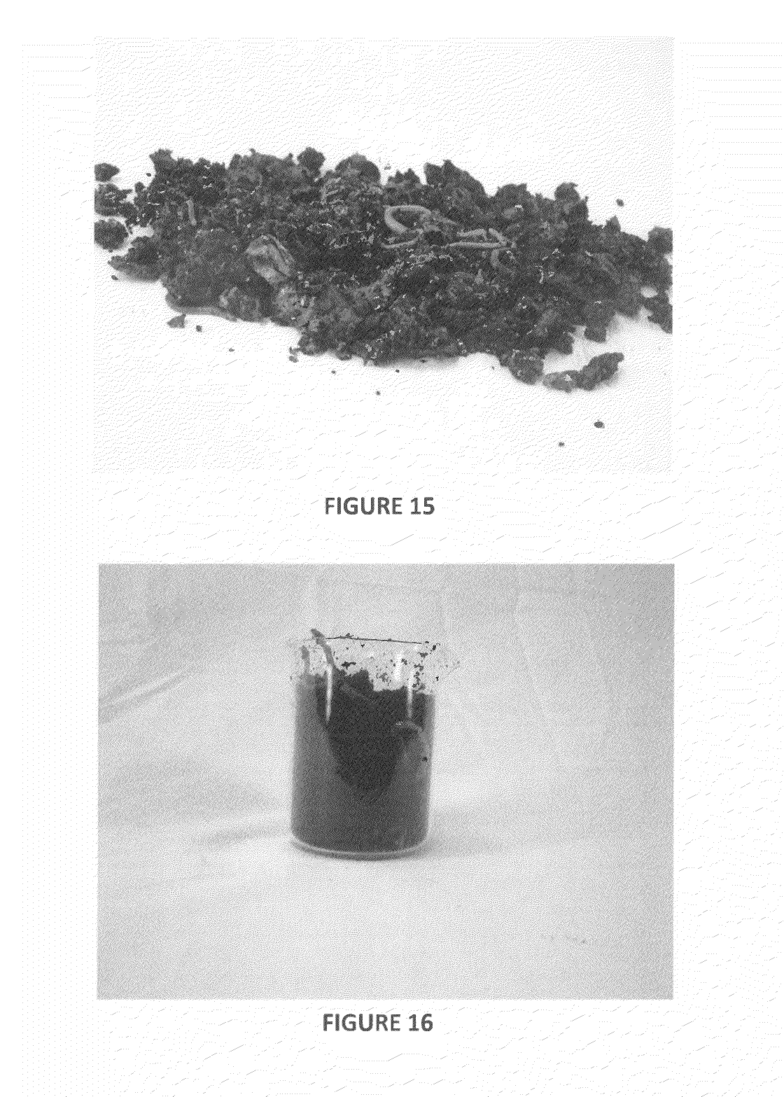

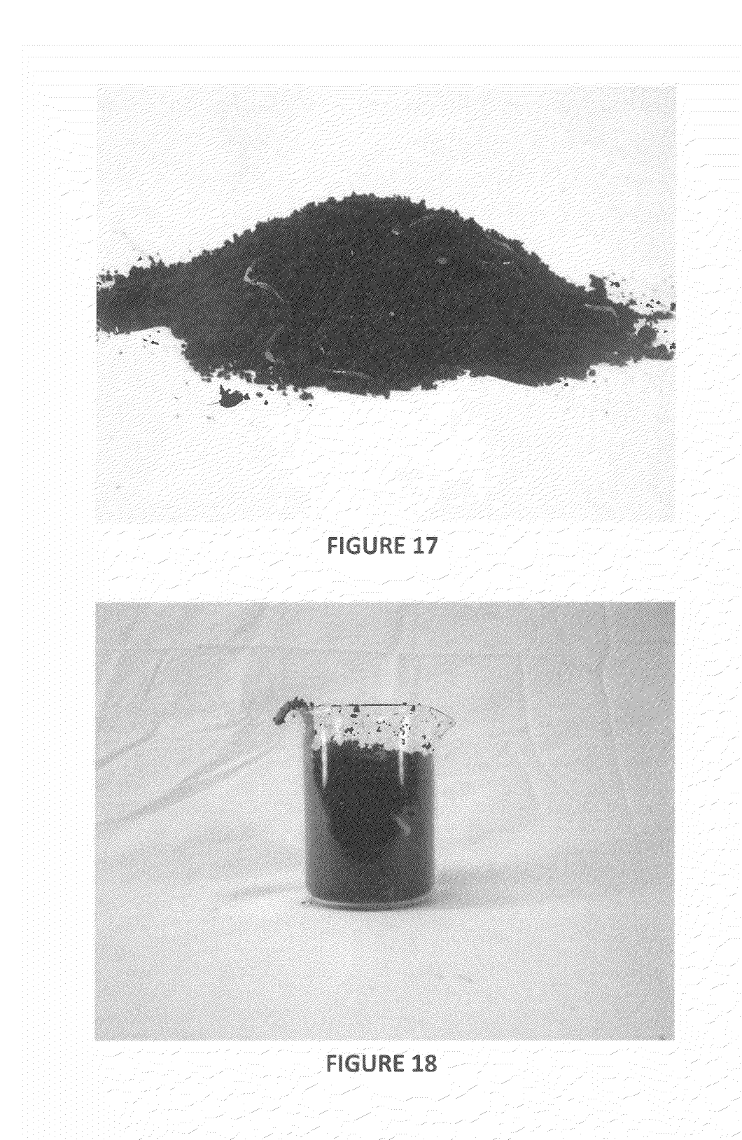

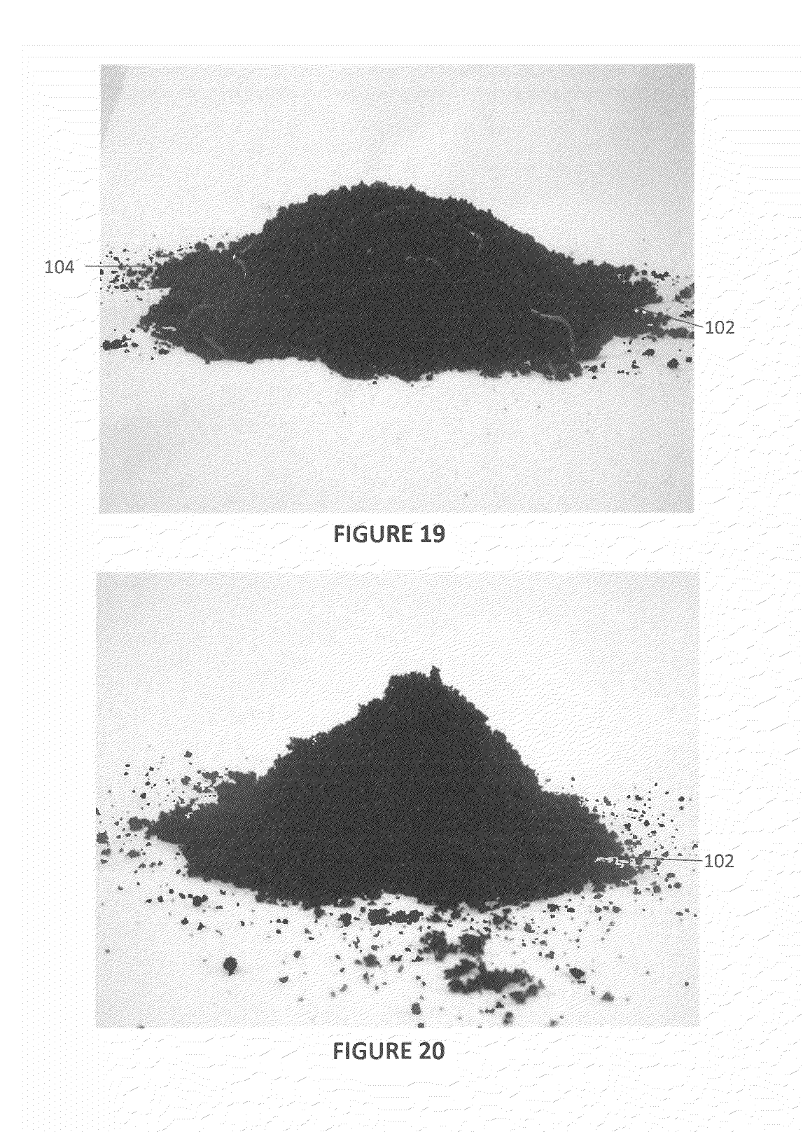

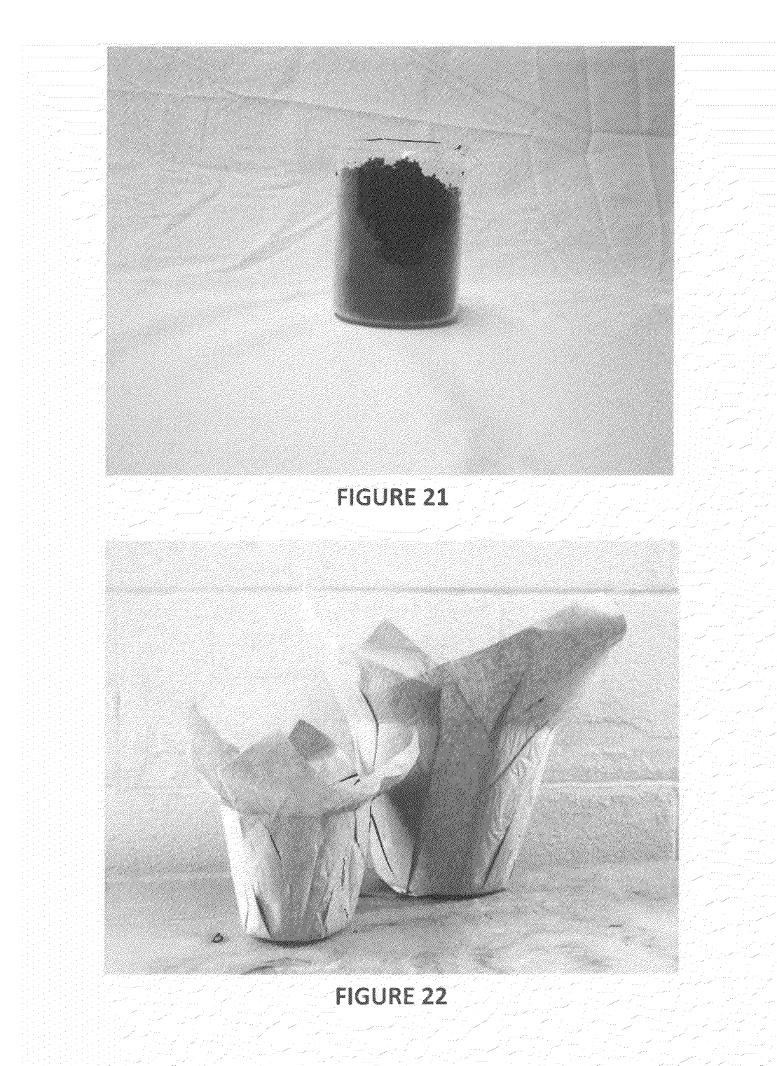

FIG. 9 is a photo of one embodiment of an article (i.e., a flower pot cover, at least a portion of which is formed of paper) constructed in accordance with the presently disclosed and claimed inventive concept(s) for consumption by one or more species of earthworms. The flower pot cover is provided as the article component of any of the kits described herein above; for example but not by way of limitation, the flower pot cover may be packaged in a kit that also contains earthworm(s), or the flower pot cover may be packaged in a kit that also contains instructions for obtaining earthworm(s). FIGS. 10-21 depict disposal of soil and earthworms within the flower pot cover of FIG. 9, followed by depictions of various stages of consumption of the flower pot cover by the earthworms disposed therein. In FIGS. 20-21, the flower pot cover has been consumed and converted to soil by the earthworms. In addition, the earthworms excreted castings that added various desirable features to the soil, including but not limited to, humus, nitrogen, phosphates, potassium, plant growth hormones, and factors enhancing soil water retention properties.

### Example 2

In Example 1, earthworms were disposed in soil to expedite consumption of the article; however, it is to be understood that worms or other living organisms utilized in accordance with the presently disclosed and claimed inventive concept(s) may be placed directly in contact with the article to be consumed in the absence of any additional materials. When earthworms are utilized, the only requirement is that moisture be present. FIGS. 22-25 illustrate another embodiment of the presently disclosed and claimed inventive concept(s), in which an article obtained from a kit (produced as described herein above) is consumed by a living organism without the use of a soil or other maintenance material.

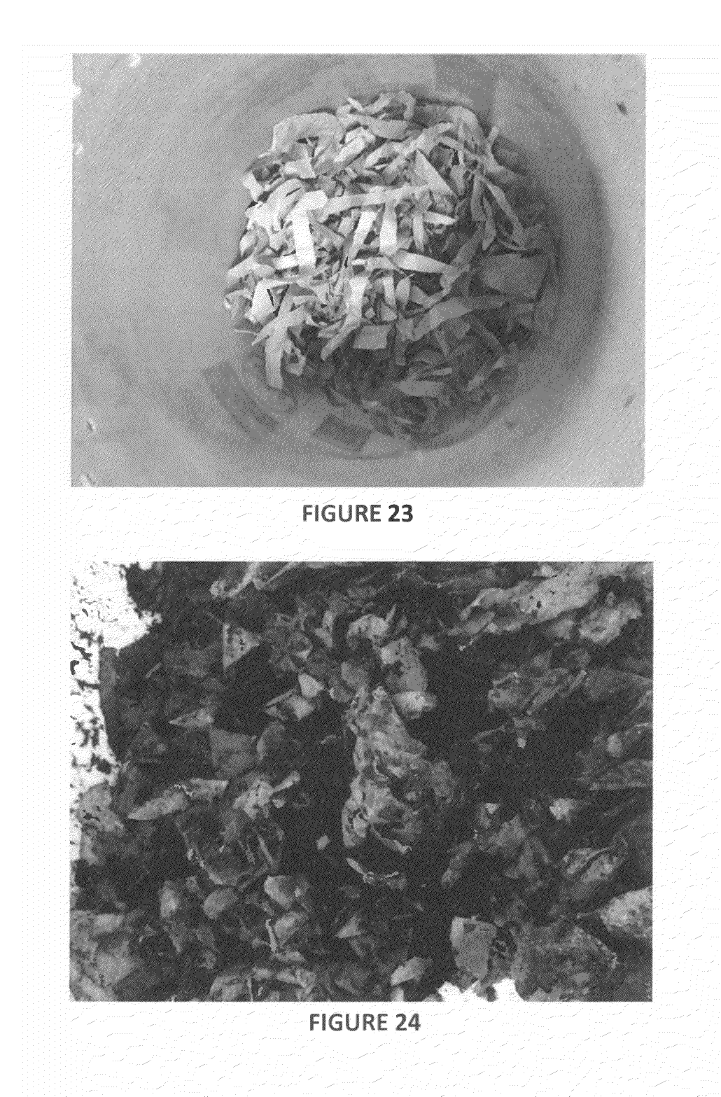

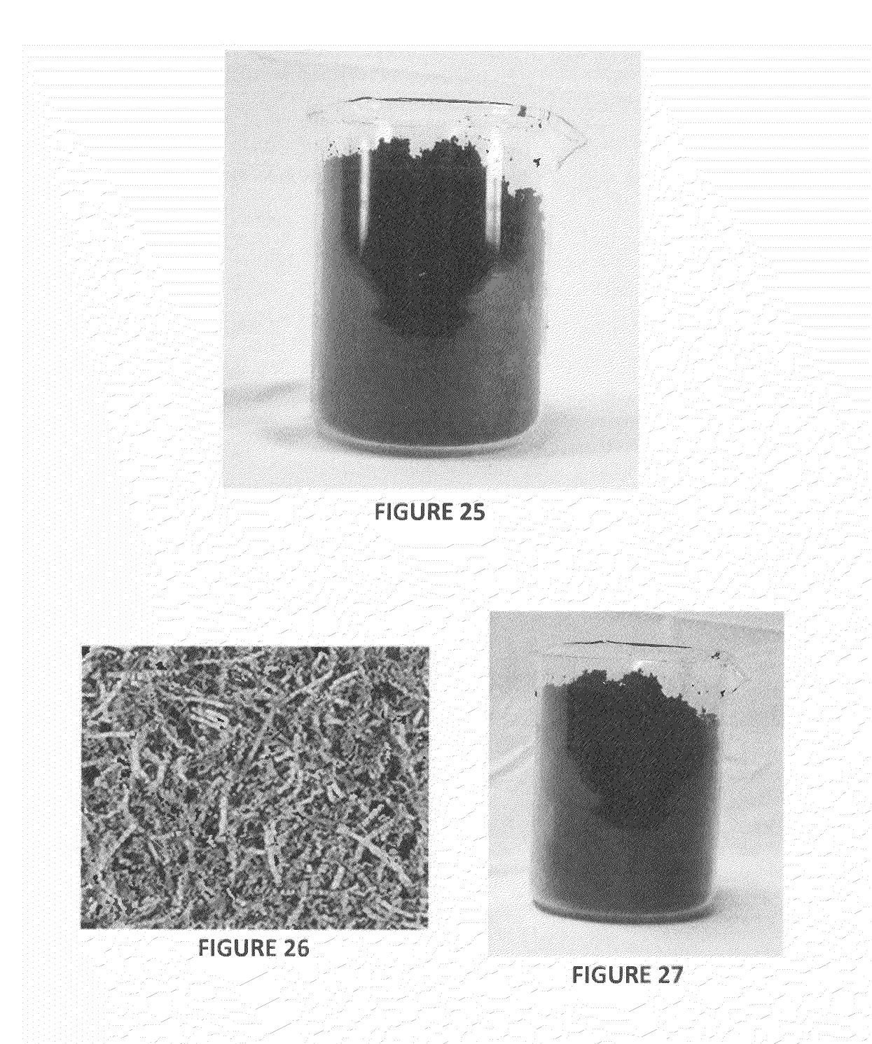

In Example 2, four paper flower pot covers similar to those shown in FIG. 22 served as the article present in a kit produced as described herein. The paper flower pot covers also contained a lacquer or other type of bonding material that connected some of the overlapping folds produced during production of the covers. The four paper flower pot covers were divided into smaller pieces/segments, as shown in FIG. 23; the segments were then moistened, and 8 ounces of earthworms of mixed species endogenous to North American soils were added to the moistened segments of covers. Within 14 days, the earthworms had consumed 50% of the material obtained from the four paper flower pot covers. FIGS. 24 and 25 depict two stages of consumption of the covers. As depicted in these figures, the earthworms consumed the flower pot cover segments and converted them to soil, which contained castings that imparted various desirable features to the soil, as described herein above in Example 1.

In another experiment, earthworms did not consume a competing flower pot cover formed from petrochemical-based films (not shown).

### Example 3

FIG. 26 illustrates consumption of another embodiment of an article (i.e., a decorative grass or packing material) obtained from any of the kits described herein above. Upon moistening the decorative grass/packing material of FIG. 26 and placing the grass/material in contact with earthworms in a manner similar to that described herein, the earthworms begin consumption of the decorative grass/packing material. FIG. 27 illustrates the results obtained by consumption of an article similar to that shown in FIG. 26 by worms in accordance with the kits and methods disclosed and claimed herein.

Thus, in accordance with the presently disclosed and claimed inventive concept(s), there has been provided kits and assemblies, as well as methods of producing and using same, that fully satisfy the objectives and advantages set forth hereinabove. Although the presently disclosed and claimed inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the presently disclosed and claimed inventive concept(s). Changes may be made in the construction and the operation of the various components, elements, and assemblies described herein, as well as in the steps or the sequence of steps of the methods described herein, without departing from the spirit and scope of the presently disclosed and claimed inventive concept(s) as defined in the following claims.

What is claimed:

1. A kit, comprising:
an article formed of a material, wherein at least a portion of the material is consumable by at least one living organism; and
at least one living organism capable of consuming the consumable portion of the material.

2. The kit of claim 1, wherein the consumable portion of the material is formed of at least one of paper and cardboard.

3. The kit of claim 1, wherein the at least one living organism is at least one of a worm, a termite, an ant, a microorganism, and combinations thereof.

4. The kit of claim 1, wherein the at least one living organism is provided in an encapsulated and/or cocooned form.

5. The kit of claim 1, wherein the at least one living organism is attached to and/or contained within the article.

6. The kit of claim 5, wherein the at least one living organism is encapsulated and/or cocooned, and the encapsulated and/or cocooned living organism is present in a coating disposed on a surface of the article.

7. The kit of claim 1, wherein the at least one living organism is not attached to the article within the kit.

8. The kit of claim 1, wherein the article is selected from the group consisting of a floral packaging material, a decorative grass, a packaging material, a cup, a bag, a food wrapper, a food container, a plate, a bowl, toilet paper, a paper towel, and combinations thereof.

9. The kit of claim 1, wherein the article is at least one of a floral packaging material, a decorative grass, a packaging material, and combinations thereof.

10. The kit of claim 1, wherein a composition has been applied to at least a portion of the article to enhance and/or expedite its consumption by the at least one living organism.

11. The kit of claim 10, wherein the composition is at least one of a nutrient, a stimulant, an attractant, a hormone, and combinations thereof.

12. The kit of claim 10, wherein the composition comprises at least one of a metal, a metal salt, a metal stearate, theobromine, theophylline, mildronate, 3-(2,2,2-trimethylhydraziniumyl)propionate, caffeine, starch, sugar, tea, tea leaves, chocolate, cocoa, coffee, coffee grounds, ground coffee beans, a short chain saturated fat, a grain, a thyroid hormone, a pheromone, dichloroacetic acid (DCA), sodium dichloroacetate, an enzyme, and combinations thereof.

13. The kit of claim 1, wherein the article is a flower pot cover and the at least one living organism is a worm, and wherein the consumable portion of the flower pot cover is formed of paper.

14. The kit of claim 1, further comprising instructions for causing the at least one living organism to begin consumption of the consumable portion of the article.

15. A kit, comprising:
an article formed of a material, wherein at least a portion of the material is consumable by at least one living organism; and
instructions for obtaining at least one living organism capable of consuming the consumable portion of the material.

16. The kit of claim 15, wherein the consumable portion of the material is formed of at least one of paper and cardboard.

17. The kit of claim 15, wherein the at least one living organism is at least one of a worm, a termite, an ant, a microorganism, and combinations thereof.

18. The kit of claim 15, wherein the at least one living organism is provided in an encapsulated and/or cocooned form.

19. The kit of claim 15, wherein the article is selected from the group consisting of a floral packaging material, a decorative grass, a packaging material, a cup, a bag, a food wrapper, a food container, a plate, a bowl, toilet paper, a paper towel, and combinations thereof.

20. The kit of claim 15, wherein the article is at least one of a floral packaging material, a decorative grass, a packaging material, and combinations thereof.

21. The kit of claim 15, wherein a composition has been applied to at least a portion of the article to enhance and/or expedite its consumption by the at least one living organism.

22. The kit of claim 21, wherein the composition is at least one of a nutrient, a stimulant, an attractant, a hormone, and combinations thereof.

23. The kit of claim 21, wherein the composition comprises at least one of a metal, a metal salt, a metal stearate, theobromine, theophylline, mildronate, 3-(2,2,2-trimethylhydraziniumyl)propionate, caffeine, starch, sugar, tea, tea leaves, chocolate, cocoa, coffee, coffee grounds, ground coffee beans, a short chain saturated fat, a grain, a thyroid hormone, a pheromone, dichloroacetic acid (DCA), sodium dichloroacetate, an enzyme, and combinations thereof.

24. The kit of claim 15, wherein the article is a flower pot cover and the at least one living organism is a worm, and wherein the consumable portion of the flower pot cover is formed of paper.

25. The kit of claim 15, further comprising instructions for causing the at least one living organism to begin consumption of the consumable portion of the article.

26. A method of degrading an article, comprising the steps of:
opening the kit of claim 1; and
exposing the article to the at least one living organism, wherein the at least one living organism consumes the consumable portion of the article.

27. A method of degrading an article, comprising the steps of:
opening the kit of claim 15;
obtaining the at least one living organism; and
exposing the article to the at least one living organism, wherein the at least one living organism consumes the consumable portion of the article.
